(19) United States
(12) Patent Application Publication
(10) Pub. No.: US 2025/0062018 A1
(43) Pub. Date: Feb. 20, 2025

(54) FREQUENT FLIER ALERT AND REPORT

(71) Applicant: Cardiac Pacemakers, Inc., St Paul, MN (US)

(72) Inventors: David L. Perschbacher, Blaine, MN (US); Keith Joseph Mattson, Lino Lakes, MN (US); Keith L. Herrmann, Minneapolis, MN (US); Derek D. Bohn, Woodbury, MN (US)

(21) Appl. No.: 18/806,121

(22) Filed: Aug. 15, 2024

Related U.S. Application Data
(60) Provisional application No. 63/533,190, filed on Aug. 17, 2023.

(51) Int. Cl. G16H 40/60

(52) U.S. Cl. CPC G16H 40/60

(57) ABSTRACT

A method includes storing, by one or more servers, separate packages of data received from separate medical devices. The separate packages of data are respectively associated with different patients. The method includes automatically determining which subset of the patients experiences the most health events among the different patients. Access to the packages of data is provided via a user interface, and an indicator is displayed on the user interface showing which of the patients is associated with the subset. A computing system such as a server can be programmed to carry out one or more steps of the method.

### CROSS REFERENCE TO RELATED APPLICATION

This application claims priority to Provisional Application No. 63/533,190, filed Aug. 17, 2023, which is herein incorporated by reference in its entirety.

### TECHNICAL FIELD

The present disclosure relates generally to approaches for assisting with efficient evaluation of health events detected by medical devices.

### BACKGROUND

Medical devices that allow physicians to monitor cardiac activity are becoming increasingly common in diagnosing and treating medical conditions in patients. Cardiac monitoring can be used, for example, to identify abnormal cardiac rhythms, so that critical alerts can be provided to patients, physicians, or other care providers and so that patients can be treated as needed.

### SUMMARY

In Example 1, a method includes storing, by one or more servers, separate packages of data received from separate medical devices. The separate packages of data are respectively associated with different patients. The method includes automatically determining which subset of the patients experiences the most health events among the different patients. Access to the packages of data is provided via a user interface, and an indicator is displayed on the user interface showing which of the patients is associated with the subset.

In Example 2, the method of Example 1, further including detecting, using the medical devices, the health events each based on a comparison of respective physiological data collected by the medical devices to one or more respective programmed parameters of the medical devices.

In Example 3, the method of any of the preceding Examples, wherein the automatically determining is based on one or more thresholds.

In Example 4, the method of Example 3, wherein the one or more thresholds includes a minimum number of detected health events.

In Example 5, the method of Example 3, wherein the one or more thresholds includes an average number of health events per day.

In Example 6, the method of Example 3, wherein the one or more thresholds includes a percentage of days with at least one health event.

In Example 7, the method of Example 3, wherein the one or more thresholds includes a percentage of total episodes within a given patient population.

In Example 8, the method of Example 3, wherein the one or more thresholds includes a number of health events over a set time period.

In Example 9, the method of any of the preceding Examples, wherein the medical devices are implantable cardiac monitors.

In Example 10, the method of any of the preceding Examples, wherein the indicator is an icon representing a high-volume patient.

In Example 11, the method of any of the preceding Examples, wherein the separate packages of data comprise an electrocardiogram plot.

In Example 12, the method of any of the preceding Examples, wherein the health event is a cardiac event.

In Example 13, a computer program product comprising instructions to cause one or more processors to carry out the steps of the method of Examples 1-12.

In Example 14, a computer-readable medium having stored thereon the computer program product of Example 13.

In Example 15, a computer comprising the computer-readable medium of Example 14.

In Example 16, a method includes storing, by one or more servers, separate packages of data received from separate medical devices. The separate packages of data are respectively associated with different patients. The method includes automatically determining which subset of the patients experiences the most health events among the different patients. Access to the packages of data is provided via a user interface, and an indicator is displayed on the user interface showing which of the patients is associated with the subset.

In Example 17, the method of Example 16, further including: detecting, using the medical devices, the health events each based on a comparison of respective physiological data collected by the medical devices to one or more respective programmed parameters of the medical devices.

In Example 18, the method of Example 17, wherein the packages of data include strips of the respective physiological data associated with the health events.

In Example 19, the method of Example 17, further including: reprogramming at least one of the medical devices associated with the subset.

In Example 20, the method of Example 16, wherein the automatically determining is based on one or more thresholds.

In Example 21, the method of Example 20, wherein the one or more thresholds includes a minimum number of detected health events.

In Example 22, the method of Example 20, wherein the one or more thresholds includes an average number of health events per day.

In Example 23, the method of Example 20, wherein the one or more thresholds includes a percentage of days with at least one health event.

In Example 24, the method of Example 20, wherein the one or more thresholds includes a percentage of total episodes within a given patient population.

In Example 25, the method of Example 20, wherein the one or more thresholds includes a number of health events over a set time period.

In Example 26, the method of Example 16, wherein the medical devices are implantable cardiac monitors.

In Example 27, the method of Example 16, wherein the indicator is an icon representing a high-volume patient.

In Example 28, system includes a server with memory and one or more processors. The memory stores instructions that, when executed, cause the server to: receive and store separate packages of data from a fleet of medical devices, automatically determine a subset of the patients that experiences the most health events among the different patients, provide access to the packages of data via an online platform, and cause an indicator to be displayed on a user interface showing which of the patients is associated with the subset. The separate packages of data are respectively associated with different patients, each indicate a health event detected by one of the medical devices, and each include data about the health event.

In Example 29, the system of Example 28, further including the fleet of medical devices. The medical devices are implantable medical devices that are programmed to detect the health events based on a comparison of respective physiological data collected by the medical devices to one or more respective programmed parameters of the medical devices.

In Example 30, the system of Example 28, further including a remote computer including a display that is arranged to display the user interface.

In Example 31, the system of Example 28, wherein the automatically determining is based on one or more thresholds.

In Example 32, the system of Example 31, wherein the one or more thresholds includes a minimum number of detected health events.

In Example 33, the system of Example 31, wherein the one or more thresholds includes an average number of health events per day.

In Example 34, the system of Example 31, wherein the one or more thresholds includes a percentage of total episodes within a given patient population.

In Example 35, the system of Example 31, wherein the one or more thresholds includes a number of health events over a set time period.

While multiple instances are disclosed, still other instances of the present disclosure will become apparent to those skilled in the art from the following detailed description, which shows and describes illustrative instances of the disclosure. Accordingly, the drawings and detailed description are to be regarded as illustrative in nature and not restrictive.

### BRIEF DESCRIPTION OF THE DRAWINGS

FIG. 1 is a schematic illustration of an implantable medical device that delivers electrical simulation to a patient's heart, in accordance with certain instances of the present disclosure.

FIG. 2 is an electrocardiogram plot, in accordance with certain instances of the present disclosure.

FIG. 3 is a summary page of detected cardiac activity, in accordance with certain instances of the present disclosure.

FIG. 4 is a block diagram illustrating a method, in accordance with certain instances of the present disclosure.

FIG. 5 is an example of a user interface, in accordance with certain instances of the present disclosure.

FIG. 6 is a block diagram depicting an illustrative computing device, in accordance with instances of the disclosure.

While the disclosure is amenable to various modifications and alternative forms, specific instances have been shown by way of example in the drawings and are described in detail below. The intention, however, is not to limit the disclosure to the particular instances described. On the contrary, the disclosure is intended to cover all modifications, equivalents, and alternatives falling within the scope of the disclosure as defined by the appended claims.

### DETAILED DESCRIPTION

Medical devices can be equipped with one or more sensing devices (e.g., sensors, electrodes) and programmed to detect potential health events experienced by a patient. For example, physiological data such as electrocardiogram (ECG) data or other types of physiological data can be used to identify whether the patient has experienced a cardiac event. To collect physiological data, one or more medical devices (e.g., implantable monitors, external monitors) can be implanted in or coupled to the patient such that the medical devices sense and record the physiological data. This physiological data can be processed and analyzed by the medical devices themselves or by a separate device such as a computing device/system (e.g., a server) to detect whether the patient experienced a health event. The detected health events may ultimately be assessed by a physician to determine whether and/or how treatment should be administered to the patient.

However, the number and frequency of detected health events can be overwhelming to process and can potentially bury the more critical health events that may require treatment. In addition, the number and frequency of detected health events may indicate that a medical device should be reprogrammed to reduce the number of detected health events that are considered false positives. Further yet, it has been found that a relatively small percentage of patients in a patient population tend to generate a relatively large percentage of the overall number of detected health events within the patient population. For example, in some patient populations, around twenty percent of the patients can account for more than eighty percent of the detected health events, and the uneven distribution can be more extreme in other patient populations. Knowing which subset of patients is generating the most detected health events can assist with more efficient processing, analyzing, and subsequent treatment of detected health events for the entire patient population.

Certain instances of the present disclosure are accordingly directed to systems, methods, and devices that assist with efficient evaluation of detected health events.

### Health Event Detection and Evaluation System

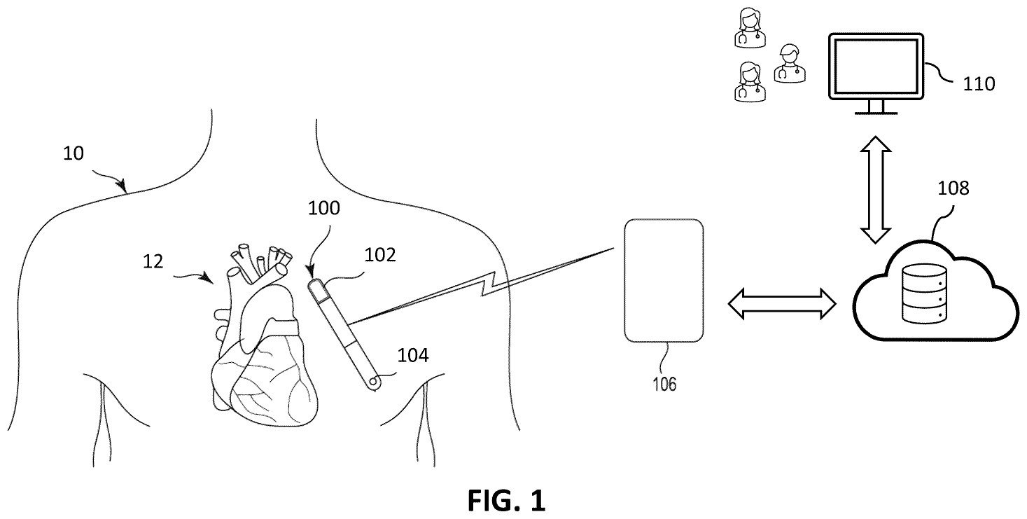

FIG. 1 is a schematic illustration of a medical device 100 within a health event detection and evaluation system. The medical device 100 can be implanted subcutaneously within an implantation location or pocket in the patient's chest or abdomen and may be configured to monitor (e.g., sense and/or record) physiological signals associated with the patient's heart 12. The medical device 100 may be an implantable cardiac monitor (e.g., an implantable diagnostic monitor, an implantable loop recorder) configured and programmed to record physiological parameters such as, for example, one or more cardiac activation signals, heart sounds, blood pressure measurements, oxygen saturations. Although the medical device 100 of FIG. 1 is shown as an implantable cardiac monitor, the approaches described herein can be used in connection with other types of implantable medical devices such as a pulse generator (e.g., pacemaker, defibrillator). Further, although the medical device 100 of FIG. 1 is shown as a medical device that is implanted into the patient 10, the approaches described herein can be used in connection with an external medical device such as a monitoring device that is attached to a patient's skin or a mobile device that includes one or more sensors for detecting physiological data and/or that can process detected physiological data. Further yet, although the medical device 100 is described as a device that detects physiological data related to cardiac activity, the approaches described herein can apply to other types of physiological data which is used by physicians to determine whether treatment should be administered to the patient 10.

In the example of FIG. 1, the medical device 100 includes electrodes 102 and 104, which sense physiological signals (e.g., electrical signals) of the heart 12. The medical device 100 can record responsive physiological data to local memory and periodically communicate (e.g., communicate wirelessly) the recorded data to a receiver 106 (e.g., programmer, controller, patient monitoring system, mobile phone, computer external to the patient). The receiver 106 can be communicatively coupled to the medical device 100 and positioned external to the patient's body. The receiver 106 can be a device that is capable of programming, controlling, monitoring, and/or otherwise communicating with the medical device 100. The receiver 106 can help facilitate communication from the medical device 100 to another device or system such as a computing system 108 (e.g., laptop computer, desktop computer, server). The receiver 106 and/or the computing system 108 can be communicatively coupled to another computing system 110 with a display on which users (e.g., patients, physicians, technicians) can view data generated by the medical device 100.

In certain instances, the medical device 100 is programmed to detect potential health events such as potential cardiac events (e.g., arrhythmias or other types of cardiac episodes) based on the physiological data sensed by the medical device 100. For example, the medical device 100 can compare the physiological data to various programmed parameters (e.g., thresholds for items such as event duration, heart rate) and, if one or more parameters are met (e.g., thresholds breached), the medical device 100 can determine that a potential health event has occurred.

If the medical device 100 is configured to provide therapy (e.g., an electrical stimulation to the heart 12), the medical device 100 can deliver such therapy in response to the physiological data and the detected potential health event. For example, the medical device 100 can deliver certain therapy based on how the medical device 100 is programmed to respond to the physiological data and the detected potential health event.

In certain instances, the recorded physiological data (and associated metadata) may be downloaded from one or more of the medical device 100 and the receiver 106 periodically or on command. Further, the physician and/or the patient 10 may communicate with the medical device 100 and the receiver 106, for example, to acquire data or to initiate, terminate, or modify (e.g., reprogram) sensing and/or therapy parameters.

The programmed parameters can include a wide variety of parameters. For example, a medical device 100 may have 20-30 parameters that can be set and adjusted based on a given patient's history and a physician's recommendations. The parameters can relate to how the medical device 100 monitors the patient's physiological data. For example, the parameters can include sensing-related parameters (e.g., thresholds, values) that are set to—individually or in combination—detect when a patient has potentially experienced a health event. As another example, the parameters can include therapy-related parameters (e.g., thresholds, output values, and action commands) that are set to—individually or in combination—dictate if, when, and how therapy is provided by the medical device 100 after a potential health event has been detected.

When a potential health event is detected (based on one or more programmed parameters), the medical device 100 can begin to store data associated with the detected health event to local memory. In some instances, the medical device 100 is configured to repeatedly delete a certain amount of historical sensed physiological data until a health event is detected. For example, the medical device 100 may continuously delete historical data (that is not associated with a detected health event) after expiration of a rolling period of time (e.g., 10-20 seconds) to preserve memory capacity. However, once a potential health event is detected, the medical device 100 can begin storing physiological data until the health event is determined to be complete. This may include storing any physiological data that had not already been deleted as well as a certain amount of physiological data sensed after completion of the detected health event (e.g., 20-60 seconds of physiological data).

As a result, when a potential health event is detected, the medical device 100 may store physiological data associated with the detected health event, including a certain amount of physiological data before and after the onset and completion of the detected health event. In certain instances, completion of the detected health event can be determined once the medical device 100 has not detected an abnormal heartbeat (e.g., as defined by threshold values) after a certain window of time (e.g., a window of 10-60 seconds). This window of time can be different depending on whether therapy was delivered. For example, if a potential health event was detected but did not result in therapy being delivered, the window of time can be less than if the potential health event resulted in therapy. The medical device 100 can also be programmed to record metadata associated with the detected health event. Example metadata can include durations associated with the cardiac event/subevents, heart rate, type of health event, therapy information (e.g., charge time, lead impedance, lead polarity, ATP scheme), etc.

### Health Event Evaluation

As noted above, the detected health events may ultimately be evaluated by a physician to determine whether and/or how treatment should be administered to the patient and/or whether the medical device 100 should be reprogrammed. To facilitate evaluation, a user can access patients' health event information via the computing system 110 and view the information on the display. For example, after the physiological data and metadata associated with detected health events is transmitted from the medical device 100, the computing system 108 such as one or more servers (hereinafter the “server 108” for brevity, although other types of computing systems can be used in place of a server or in addition to a server) can provide selective access to such data via the computing system 110. In certain instances, the server 108 hosts a software-as-a-service (SaaS) platform (e.g., an online platform), which can be accessed by approved users via a web browser on the computing system 110.

Depending on the size of a clinic, there may be dozens, hundreds, or even thousands of patients with medical devices programmed to detect potential health events and transmit associated data to the server 108. As such, for just one clinic, the server 108 may store hundreds, thousands, or millions of separate packages of data—each associated with a different detected health event.

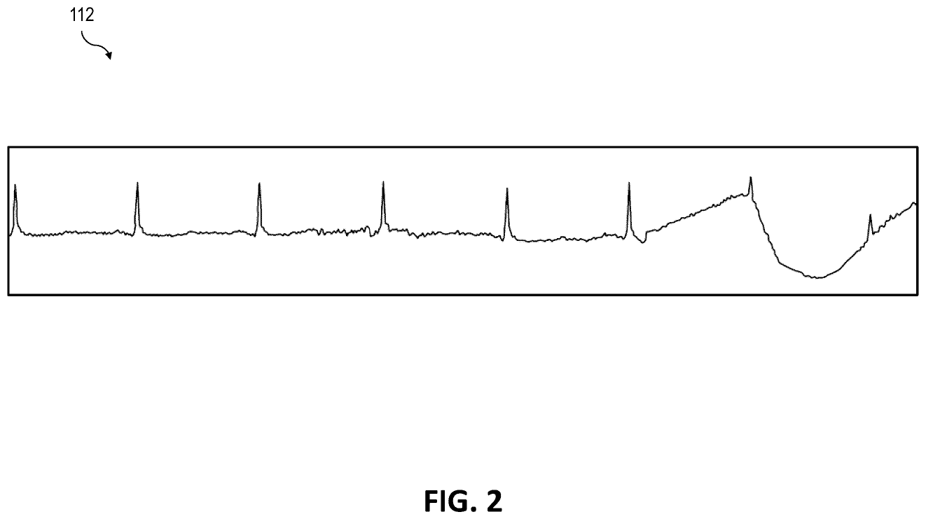

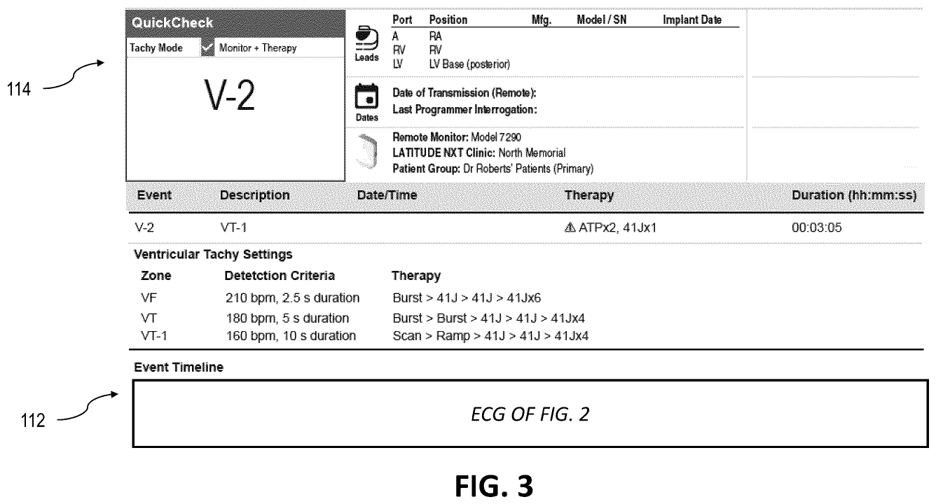

FIGS. 2 and 3 show examples of the types of health-event-related information that a clinic can access from the server 108 via the computing system 110. FIG. 2 shows a short strip 112 of ECG data. As noted above, when the medical device 100 detects a potential cardiac event, the medical device 100 can begin to record physiological data. As such, each detected cardiac event may be associated with one or more strips of ECG data-all of which can be stored to the server 108 and accessed at the computing system 110. Each clinic can include more than one computing system 110, and many different clinics can access information about their respective patients from the server 108.

In addition to the underlying ECG data, the clinic can access a summary of each detected cardiac event. FIG. 3 shows an example summary report 114 that includes metadata that describes aspects of a detected cardiac event. The report 114 can also include the underlying strips 112 of ECG data.

Although the strips 112 of ECG data and reports 114 are useful in evaluating whether a cardiac event warrants treatment, the number and frequency of detected cardiac events can be overwhelming to process such information for each detected health event. As noted above, it has been found that a relatively small percentage of patients in a patient population tend to generate a relatively large percentage of the overall number of detected health events within the patient population. Knowing which patients are generating the majority of detected health events can assist with more efficient processing, analyzing, and subsequent treatment of detected health events.

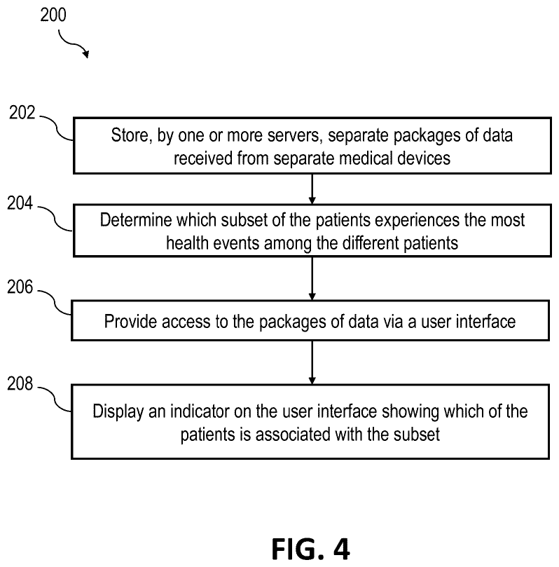

FIG. 4 outlines steps of a method 200 for facilitating such evaluation. The method 200 includes storing separate packages of data (e.g., by a server) each associated with a health event detected by a medical device (block 202 in FIG. 4). The medical device can be part of a fleet of medical devices prescribed to different patients associated with a clinic. One or more packages of data can be associated with a different patient within the clinic's patient population. Further, each package of data can include data (e.g., physiological data and metadata) about a given health event. The device or system storing the packages of data can store such data for multiple clinics, which can access the data via remote devices.

The method 200 further includes determining which subset of the patients experiences the most health events among the entire patient population (block 204 in FIG. 4). In certain instances, determining which patients experience the most health events (and therefore generate the most data) is done automatically by using one or more thresholds involving the number of detected health events for a given patient. Example thresholds include: the average number of health events per day, the percentage of days with at least one health event, the overall total number of health events over time, the percentage of total episodes within a given patient population, the number of health events over a set time period (e.g., 7-day period, 14-day period, 30-day period), and/or rates of change (e.g., increase of 20% or more) for any of the above-noted thresholds.

Thresholds can be set such that a patient is identified as a “high-volume” patient or a “frequent-flier” patient if a patient experiences more detected health events than one or more of the set thresholds. In certain instances, patients are placed into different levels/grades of patients identified as being high-volume patients-instead of the high-volume patient classification being a binary classification. In such examples, the thresholds can be ranges, where each separate range is associated with a different level or grade indicating a degree or severity of high-volume patient.

In certain instances, the patient must have a minimum of two days' worth of monitoring before a patient is eligible to be identified as a high-volume patient. Further, the process of determining which subset of the patients experiences the most health events among a patient population can be rerun and confirmed over time. For example, after a certain period of time (e.g., multiple days, a week, a month, and the like), the patient's health event activity (e.g., most recent health event activity) can be assessed and determined whether the patient should remain identified as a high-volume patient or should be associated with a different level/grade. As another example, a patient's status as a high-volume patient may be reassessed after the patient's medical device is reprogrammed (e.g., to include less sensitive thresholds to reduce the number of potential events detected by the medical device).

In certain instances, determining the subset of patients that are high-volume patients is performed on a clinic-by-clinic basis. As such, each clinic may set its own thresholds to determine which patients are flagged as high-volume patients. Further, the health event activity of a given patient (e.g., number of health events, percentage of health events) may be compared to other patients associated with the same clinic.

The method 200 further includes providing access to the packages of data via a user interface (block 206 in FIG. 4). For example, the user interface can display features such as those shown in FIGS. 2 and 3. The user interface can be shown on a display (e.g., monitor, screen).

The method 200 further includes displaying an indicator on the user interface showing which of the patients is associated with the subset (block 208 in FIG. 4).

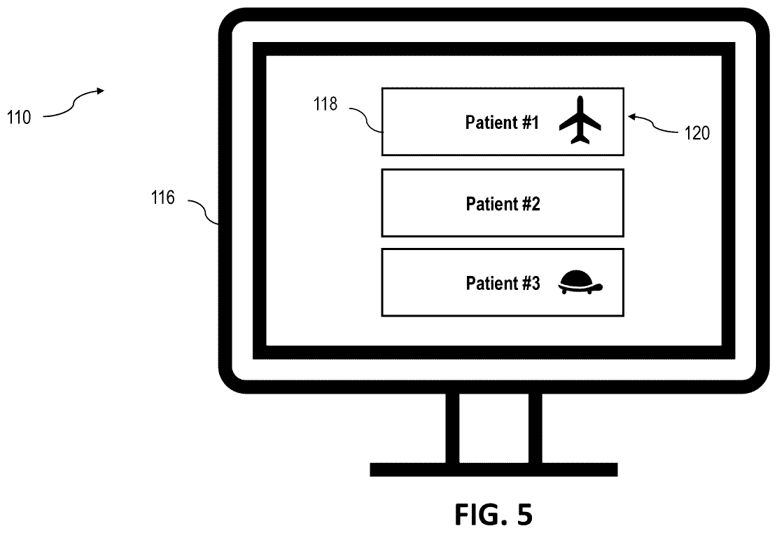

FIG. 5 shows the computing system 110 having a display 116, which displays a user interface. The user interface can include a page that lists different patients that have experienced a potential health event. Each patient can be represented by a graphic 118, which includes information such as the patient's name and other types of information about the patient such as the number of detected health events, among other things. For patients determined to be high-volume patients, the user interface can display an indicator 120 that denotes that the patient is a high-volume patient. The indicator 120 can be an icon/graphic/text that helps the user distinguish between regular patients and high-volume patients. In the example of FIG. 5, the indicator is an airplane icon-however, text, graphics, and/or other icons can be used for the indicator 120. In certain instances, the indicator 120 (e.g., via color) or a separate indicator can denote trends of the patient's detected health events such as whether there is a change in the rate (e.g., decreasing or increasing number of detected health events per day) of health events experienced by the patient.

Denoting high-volume patients can assist with efficient processing, evaluating, and potentially prescribing treatment for detected health events in multiple ways.

As one example, for patients that are truly experiencing a high volume of health events such as cardiac episodes, the person tasked with evaluating the cardiac episodes can focus attention on the most critical cardiac episodes. The most critical cardiac episodes may be the episodes with longer durations, faster heart rate, longer R-R intervals. The user interface can be used to sort the detected health events for a given patient by duration, heart rate, R-R interval, etc., so that such events can be easily identified.

As another example, the volume of detected potential health events may indicate that the patient's medical device should be reprogrammed. As noted above, medical devices can be programmed to detect health events by comparing sensed physiological data to programmed physiological parameters (e.g., thresholds). However, the initial programming of medical devices may need to be updated if the medical devices are not performing as expected in the field. If the programmed thresholds are set such that the medical device is perceiving non-events as events, etc., the medical device may need to be reprogrammed with updated thresholds or other types of parameters.

In certain instances, patients who have not been identified as a high-volume patient can be associated with an indicator. Denoting non-high-volume patients can assist with quickly identifying which patients—who may infrequently have health events—have experienced a potential health event such that those events can be prioritized for further assessment.

### Computing Devices and Systems

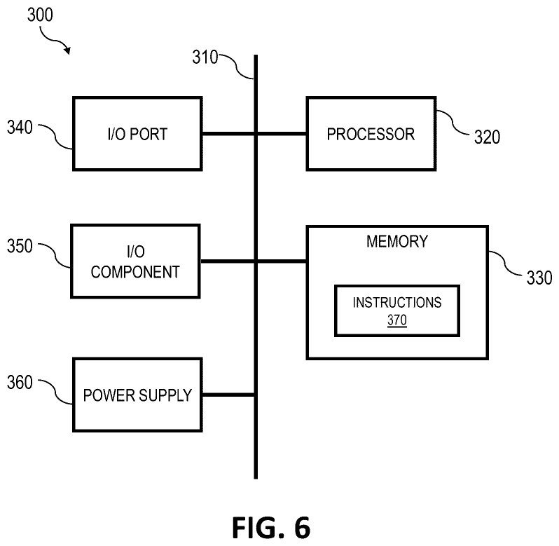

FIG. 6 is a block diagram depicting an illustrative computing device 300, in accordance with instances of the disclosure. The computing device 300 may include any type of computing device suitable for implementing aspects of instances of the disclosed subject matter. Examples of computing devices include specialized computing devices or general-purpose computing devices such as workstations, servers, laptops, desktops, tablet computers, hand-held devices, smartphones, general-purpose graphics processing units (GPGPUs), and the like. Each of the various components shown and described in the Figures can contain their own dedicated set of computing device components shown in FIG. 6 and described below. For example, the mobile devices, servers, and remote computers can each include their own set of components shown in FIG. 6 and described below.

In instances, the computing device 300 includes a bus 310 that, directly and/or indirectly, couples one or more of the following devices: a processor 320, a memory 330, an input/output (I/O) port 340, an I/O component 350, and a power supply 360. Any number of additional components, different components, and/or combinations of components may also be included in the computing device 300.

The bus 310 represents what may be one or more busses (such as, for example, an address bus, data bus, or combination thereof). Similarly, in instances, the computing device 300 may include a number of processors 320, a number of memory components 330, a number of I/O ports 340, a number of I/O components 350, and/or a number of power supplies 360. Additionally, any number of these components, or combinations thereof, may be distributed across a number of computing devices.

In instances, the memory 330 includes computer-readable media in the form of volatile and/or nonvolatile memory and may be removable, nonremovable, or a combination thereof. Media examples include random access memory (RAM); read only memory (ROM); electronically erasable programmable read only memory (EEPROM); flash memory; optical or holographic media; magnetic cassettes, magnetic tape, magnetic disk storage or other magnetic storage devices; data transmissions; and/or any other medium that can be used to store information and can be accessed by a computing device. In instances, the memory 330 stores computer-executable instructions 370 for causing the processor 320 to implement aspects of instances of components discussed herein and/or to perform aspects of instances of methods and procedures discussed herein. The memory 330 can comprise a non-transitory computer readable medium storing the computer-executable instructions 370.

The computer-executable instructions 370 may include, for example, computer code, machine-useable instructions, and the like such as, for example, program components capable of being executed by one or more processors 320 (e.g., microprocessors) associated with the computing device 300. Program components may be programmed using any number of different programming environments, including various languages, development kits, frameworks, and/or the like. Some or all of the functionality contemplated herein may also, or alternatively, be implemented in hardware and/or firmware.

According to instances, for example, the instructions 370 may be configured to be executed by the processor 320 and, upon execution, to cause the processor 320 to perform certain processes. In certain instances, the processor 320, memory 330, and instructions 370 are part of a controller such as an application specific integrated circuit (ASIC), field-programmable gate array (FPGA), and/or the like. Such devices can be used to carry out the functions and steps described herein.

The I/O component 350 may include a presentation component configured to present information to a user such as, for example, a display device, a speaker, a printing device, and/or the like, and/or an input component such as, for example, a microphone, a joystick, a satellite dish, a scanner, a printer, a wireless device, a keyboard, a pen, a voice input device, a touch input device, a touch-screen device, an interactive display device, a mouse, and/or the like.

The devices and systems described herein can be communicatively coupled via a network, which may include a local area network (LAN), a wide area network (WAN), a cellular data network, via the internet using an internet service provider, and the like.

Aspects of the present disclosure are described with reference to flowchart illustrations and/or block diagrams of methods, devices, systems and computer program products. It will be understood that each block of the flowchart illustrations and/or block diagrams, and combinations of blocks in the flowchart illustrations and/or block diagrams, can be implemented by computer program instructions.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A method comprising:
storing, by one or more servers, separate packages of data received from separate medical devices, wherein the separate packages of data are respectively associated with different patients, wherein the packages of data each indicate a health event detected by one of the medical devices, wherein the packages of data each include data about the health event;
automatically determining a subset of the patients that experiences the most health events among the different patients;
providing access to the packages of data via a user interface; and
displaying an indicator on the user interface showing which of the patients is associated with the subset.

2. The method of claim 1, further comprising:
detecting, using the medical devices, the health events each based on a comparison of respective physiological data collected by the medical devices to one or more respective programmed parameters of the medical devices.

3. The method of claim 2, wherein the packages of data include strips of the respective physiological data associated with the health events.

4. The method of claim 2, further comprising:
reprogramming at least one of the medical devices associated with the subset.

5. The method of claim 1, wherein the automatically determining is based on one or more thresholds.

6. The method of claim 5, wherein the one or more thresholds includes a minimum number of detected health events.

7. The method of claim 5, wherein the one or more thresholds includes an average number of health events per day.

8. The method of claim 5, wherein the one or more thresholds includes a percentage of days with at least one health event.

9. The method of claim 5, wherein the one or more thresholds includes a percentage of total episodes within a given patient population.

10. The method of claim 5, wherein the one or more thresholds includes a number of health events over a set time period.

11. The method of claim 1, wherein the medical devices are implantable cardiac monitors.

12. The method of claim 1, wherein the indicator is an icon representing a high-volume patient.

13. A system comprising:
a server comprising memory and one or more processors, wherein the memory stores instructions that, when executed, cause the server to:
receive and store separate packages of data from a fleet of medical devices, wherein the separate packages of data are respectively associated with different patients, wherein the packages of data each indicate a health event detected by one of the medical devices, wherein the packages of data each include data about the health event,
automatically determine a subset of the patients that experiences the most health events among the different patients,
provide access to the packages of data via an online platform, and
cause an indicator to be displayed on a user interface showing which of the patients is associated with the subset.

14. The system of claim 13, further comprising:
the fleet of medical devices, wherein the medical devices are implantable medical devices that are programmed to detect the health events based on a comparison of respective physiological data collected by the medical devices to one or more respective programmed parameters of the medical devices.

15. The system of claim 13, further comprising:
a remote computer including a display that is arranged to display the user interface.

16. The system of claim 13, wherein the automatically determining is based on one or more thresholds.

17. The system of claim 16, wherein the one or more thresholds includes a minimum number of detected health events.

18. The system of claim 16, wherein the one or more thresholds includes an average number of health events per day.

19. The system of claim 16, wherein the one or more thresholds includes a percentage of total episodes within a given patient population.

20. The system of claim 16, wherein the one or more thresholds includes a number of health events over a set time period.
